(19) United States
(12) Patent Application Publication
(10) Pub. No.: US 2012/0034604 A1
(43) Pub. Date: Feb. 9, 2012

(54) USE OF BASIC PROLIN-RICH LACRIMAL GENE PRODUCTS, SUCH AS OPIORPHIN, AS A BIOMARKER

(75) Inventors: Catherine Rougeot, Chevreuse (FR); Evelyne Dufour, Vanves (FR); Sylvie Villard-Saussine, Issy Les Moulineaux (FR); Marie-Noelle Ungeheuer, Maurepas (FR); Pierre Jouannet, Paris (FR)

(73) Assignee: INSTITUT PASTEUR, Paris Cedex 15 (FR)

(21) Appl. No.: 13/131,470

(22) PCT Filed: Nov. 27, 2009
(86) PCT No.: PCT/EP2009/066002

§ 371 Date: Sep. 6, 2011

(30) Foreign Application Priority Data
Nov. 28, 2008 (CA) 2645452

(51) Int. Cl. C12Q 1/68; C07K 1/14; C07K 16/18; G01N 33/566; C12N 5/16

(52) U.S. Cl. 435/6.11; 435/7.93; 435/331; 530/387.9

(57) ABSTRACT

The present invention relates to the use of Basic Prolin-rich Lacrimal protein (BPLP) gene products, such as Opiorphin, for establishing a prognosis, a diagnosis or the monitoring of a pathological state or of treatment efficacy in a subject and the related method of use.

### FIELD OF THE INVENTION

The present invention relates to Basic Prolin-rich Lacrimal Protein (BPLP) gene products, such as Opiorphin, and more particularly to the use of BPLP gene products as a biomarker for prognosis, diagnosis or monitoring of a pathological state or of treatment efficacy in a subject, and the related methods of use.

### BRIEF DESCRIPTION OF THE PRIOR ART

The convergent data obtained from integrative post-genomic, biochemistry and pharmacological approaches provide evidence for the existence in mammals of a physiological antagonist of NEP (Neutral EndoPeptidase, Neprilysin) and AP-N (Aminopeptidase-N) ecto-enkephalinases. The inhibitors were characterized firstly in rat (QHNPR-peptide) and named Sialorphin (as described in WO 90/03981, WO 98/00956, WO 01/00221, WO 02/041434), and more recently in Human (QRFSR-peptide) and called Opiorphin (as described in WO 05/090386).

Sialorphin is an exocrine and endocrine peptide-signal. It is an inhibitor of pain perception and acts by potentiating endogenous μ- and δ-opioid receptor-dependent enkephalinergic pathways. Its expression is under activational androgenic regulation and its secretion is evoked under adrenergic-mediated response to environmental stress in male rat (Rougeot et al., 1997, Am. J. Physiol. 273 (4pt2), R1309-1320).

Sialorphin recognizes specific target sites in organs that are deeply involved in the mineral ion concentration and thus may have a therapeutic role in the metabolic disorders related to a mineral ion imbalance, such as bone, teeth, renal, kidney intestine, pancreas, stomach mucosa or parathyroid disorders caused principally by a mineral ion imbalance in the body fluids or tissues. Accordingly, Sialorphin may be used for preventing or treating diseases like hyper- or hypo-parathyroidism, osteoporosis, pancreatitis, submandibular gland lithiasis, nephrolithiasis or osteodystrophy (WO 98/37100).

Sialorphin may also be used for the treatment of DSM disorders (see the “Diagnostic and Statistical Manual of Mental Disorders” (DSM), American Psychiatric Assoc., in particular DSM-III (1980), DSM-III-R (1987), DSM-IV (1994) and DSM-IV-TR (2000)), such as impaired interpersonal and behavioral disorders, including sexual disorders such as male erectile dysfunction (M.E.D.) and hypoactive sexual desire disorder (H.S.D.D.), by providing improved awareness and alertness to environment, improved adaptation to environment and ability to sustain attention, and increased interest in environment and capacity for arousal, without increased aggressiveness (WO 01/00221).

Sialorphin acts as natural modulator, in particular as an inhibitor, of membrane metallopeptidases such as NEP and APN. Accordingly, Sialorphin may be used for preventing or treating diseases like atherosclerosis, pain, tumors, cancers and infections (WO 021051435).

Opiorphin is considered the functional human homologous of Sialorphin. It derives from the BPLP protein (“Basic Prolin-rich Lacrimal Protein”) (WO 05/090386, Rougeot et al.). The gene BPLP is mainly expressed in human lacrimal and submandibular glands.

Opiorphin is a QRFSR peptide that inhibits two enkephalin-catabolizing ectoenzymes, human neutral ecto-endopeptidase, hNEP (EC 3.4.24.11), and human ecto-aminopeptidase, hAP-N (EC 3.4.11.2). Opiorphin displays potent analgesic activity in chemical and mechanical pain models by activating endogenous opioid-dependent transmission. The pain-suppressive potency of Opiorphin is as effective as morphine in the behavioral rat model of acute mechanical pain, the pin-pain test. Opiorphin is involved in the enkephalin-related activation of endogenous opioid-dependent pathways, which is associated with the regulation of mood-related states and pain sensation. Furthermore, because of its in vivo properties, Opiorphin may have therapeutic implications as a potential initiator of molecular pathways that could be exploited to develop new candidate drugs for the clinical management of pain relief and the alleviation of emotional disorders (Wisner et al., PNAS, 103 (47): 17979-17984 (2006)). Opiorphin may also be used for the prevention or treatment of any hydro-mineral imbalance, including disorders such as bone, teeth, kidney, parathyroid, pancreas, intestine, stomach mucosa, and salivary gland disorders. The disorder may be hyper- or hypo-parathyroidism, osteoporosis, pancreatitis, submandibular gland lithiasis, nephrolithiasis or osteodystrophy (WO 05/090386).

In order to make a prognostic, a diagnostic, or to evaluate the evolution of a pathological state including the efficiency of a treatment in a subject, there is a need in the art for biomarkers for pathological state, such as pain syndrome, hydromineral imbalance and socio-relational disorders.

### SUMMARY

The present invention provides the use of a BPLP gene product, for prognosis, diagnosis or monitoring of a pathological state in a subject. The BPLP gene product may be used as a biomarker.

The present invention is drawn to a method for prognosis, diagnosis or monitoring of a pathological state in a subject using a BPLP gene product.

The method comprises the steps of:

a) measuring the quantitative level of a BPLP gene product in a biological sample obtained from the subject;

b) comparing the quantitative level of the BPLP gene product measured in step a) to a reference value of BPLP gene product;

wherein a significantly higher or lower level of the BPLP gene product compared to the reference value is an indication for the prognosis, diagnosis or evolution of the pathological state.

The present invention also provides a kit for a prognostic, diagnostic or monitoring test. The kit comprises:
a binding agent for specifically recognizing a BPLP gene product,
reagents to detect the binding of the agent with the BPLP gene product, and
optionally, positive and/or negative control sample(s).

### BRIEF DESCRIPTION OF THE FIGURES

FIG. 1 describes the competitive ELISA immuno-assay method used for the quantification of Human Opiorphin.

FIG. 2 describes representative Opiorphin-ELISA dose-response curves. (X axis: QRFSR-peptide concentration in log scale; Y axis: percentage of specific binding (B) in the presence of standard peptide/specific binding (B0) in absence of the standard peptide).

FIG. 3 describes the specificity of the immuno-assay for Opiorphin (square) compared to Sialorphin (lozenge).

FIG. 4 describes representative Opiorphin-ELISA response curves in the saliva from healthy human volunteers (black square: standard QRFSR-peptide (Opiorphin); lozenge: Saliva-Extract n °1a-2 av (basal secretion); circle: Saliva-Extract n °1a-2 ap (stimulated secretion)).

FIG. 5 describes the quantification of Opiorphin in male saliva (1st lane, basal secretion; 2nd lane: stimulated secretion; differences between both groups: P<0.001 by Mann-Whitney test) and in female saliva (3rd lane, basal secretion; 4th lane: stimulated secretion. Statistical analysis between groups by Kruskal-Wallis test (KWT) variance analysis test: P<0.0001). Difference between both male and female saliva groups under stimulated salivary secretion conditions: P<0.005 by Mann-Whitney test. Each box represents the whole distribution of values and the inner line indicates the median value.

FIG. 6 describes the quantification of Opiorphin in male and female saliva wherein one female saliva sample is excluded from the statistical test compared to the statistical analysis in FIG. 5. (1st lane: basal conditions—men; 2nd lane: stimulated conditions—men; 3rd lane: basal conditions—women; 4th lane: stimulated conditions—women). Statistical analysis between groups by Kruskal-Wallis test (KWT) variance analysis test: P<0.0001.Statistical analysis between two groups by Mann-Whitney test: **P<0.01; ***P<0.001.

FIG. 7 describes the HPLC chromatographic elution profile and Opiorphin-ELISA analysis of two human plasma extracts: n °1a-11 and 1a-12.

FIG. 8 describes the quantification of Opiorphin in the seminal fluid from human volunteers in function of the extraction conditions: in absence or in the presence of 0.1N HCl or 1 mM EDTA plus 0.1N HCl. (analysis of variance between groups by KWT: P=0.0004; and difference between two groups by Mann-Whitney U-test: **P<0.01).

FIG. 9 describes the quantification of Opiorphin in the seminal fluid from human volunteers. (1st lane: healthy volunteers; 2nd lane: volunteers with congenital bilateral agenesis).

FIG. 10 describes the HPLC chromatographic elution profile and Opiorphin-ELISA analysis of two human sperm extracts: n °3a-13 (healthy) and 3b-02 (agenesis).

FIG. 11 describes representative Opiorphin-ELISA response curves in the urine from healthy human volunteers (square: standard QRFSR-peptide (Opiorphin); lozenge: urine extract n °1a-14; circle: urine extract n °1b-15).

FIG. 12 describes representative Opiorphin-ELISA response curves in the tears from human healthy volunteers (square: Standard QRFSR-peptide (Opiorphin); lozenge: tears extract n °1a-10; circle: tears extract n °1b-15).

FIG. 13 describes the quantification of Opiorphin in tears from human volunteers (1st lane: women; 2nd lane: men). Difference between both groups by Mann-Whitney U-test **P<0.01.

### DETAILED DESCRIPTION OF THE INVENTION

The endogenous opioid neuropeptides, i.e. the enkephalins, play a key role in the negative retro-control of the nociceptive transmission and in the modulation of the activity of cerebral structures governing the attention, the motivation and the adaptive balance of emotional states. The inventors of the present invention have surprisingly discovered that a Basic Prolin-rich Lacrimal Protein (BPLP) gene product (e.g. Opiorphin) is useful to protect these endogenous opioid neuropeptides from the inactivation by enkephalinases. Therefore, on a physiological point of view, BPLP gene products (e.g. Opiorphin) are advantageously useful as physiological regulators of enkephalinase activities in human. More particularly, BPLP gene products (e.g. Opiorphin) are further advantageously useful as new modulators of pathways controlling the painful perception, the emotional balances and the socio-relational behaviours.

Also, BPLP gene products (e.g. Opiorphin) as NEP inhibitor may be used as new modulators of pathways controlling hydromineral balances.

The inventors of the present invention have advantageously found that on a physiopathological point of view, BPLP gene products find a particular application for prognosis, diagnosis or monitoring of pathological states or of treatment efficacy and are useful as biological markers with potential prognostic, diagnostic or monitoring value in various pathological situations.

More specifically, the inventors have surprisingly found that Opiorphin secretory levels in various biological fluids can be readily detected and quantified, and that Opiorphin levels significantly vary depending on the physiological state and/or condition of the subject, for example basal vs. stimulated conditions, or male vs. female. In particular, statistical analyses showed that the salivary secretion of Opiorphin in healthy subjects is significantly increased under conditions of oral mucosal stimulation by citric acid compared to basal conditions. In addition, under stimulated conditions of salica secretions, Opiorphin levels in male saliva are significantly higher than in female of similar age. These variations of Opiorphin secretory levels are sufficient for being detected. Opiorphin levels were further found to be homogeneous within a given population (e.g. having a given age, gender and physiological state). Moreover, Opiorphin secretory levels can be detected not only in saliva, but also in various other biological fluids.

As a consequence, measure of Opiorphin levels can be used, e.g., for identifying whether a pathological state of a subject is linked with changes in Opiorphin circulating levels Opiorphin and/or for designing a treatment regimen for the patient.

1—BPLP Gene Products and Uses Thereof

It is therefore an aspect of the invention provides Basic Prolin-rich Lacrimal Protein (BPLP) gene products for use, for instance as a biomarker, in the prognosis, diagnosis or monitoring of a pathological state or of treatment efficacy in a subject.

In a specific embodiment, the invention aims at determining and/or confirming that a pathological state in a subject is linked with a BPLP gene product such as Opiorphin (i.e. that the pathological state is caused by abnormal production and/or secretion of said BPLP gene product, and/or that abnormal production and/or secretion of said BPLP gene product is a consequence of the pathological state). Indeed, many pathological states can be caused by various factors and/or defects potentially involved in the pathological state. It is thus required to diagnose the cause and/or the consequences of the pathological state. Thus the determination that significantly higher or lower levels of a BPLP gene product are found in a biological sample from the subject, compared with a reference value representative of BPLP gene product levels in a normal individual, allows diagnosing that the subject suffers from a pathological state linked with said BPLP gene product. In other terms, the methods and uses according to the invention allows determining whether the pathological state is linked with a change in production and/or secretion levels of said BPLP gene product, more specifically linked with a change in secretory levels of said BPLP gene product. Knowing that BPLP gene products such as Opiorphin play a role in enkephalin-mediated pathways, the methods and uses according to the invention further allow diagnosing whether the pathological sate is linked with a defect in enkephalin-mediated pathways.

Therefore an aspect of the invention provides Basic Prolin-rich Lacrimal Protein (BPLP) gene products for use, for instance as a biomarker, for determining and/or confirming that a pathological state is linked with a BPLP gene product in a subject.

It will be understood that BPLP gene products contemplated by the present invention may have a proteinic or a nucleotidic form. In the case where the contemplated BPLP gene product used as a biomarker has a nucleotidic form, such nucleotidic form may be, but not limited to, a BPLP mRNA. In the case where the contemplated BPLP gene product used as a biomarker has a proteinic form, such a proteinic form may be a BPLP peptide which may consist of the BPLP protein, a peptide derived from the BPLP protein, a BPLP precursor protein or a maturation product of said BPLP peptide.

In a preferred embodiment, the BPLP gene products according to the invention are the human BPLP protein, or a peptide or maturation product derived therefrom, or a precursor thereof.

In a most preferred embodiment, the BPLP gene product according to the invention is Opiorphin, i.e. a pentapeptide having the amino acid sequence QRFSR (SEQ ID NO: 3).

The BPLP protein contemplated by the present invention may be for instance encoded by the human BPLP gene. The human BPLP gene codes for a polypeptide sequence of 201 amino acids (with the potential signal peptide of secretion) predicted from the cDNA cloned and characterized by Dickinson et al. (Curr Eye Res. 15(4), 377-386, 1996). The BPLP gene is expressed in human lacrimal and submandibular glands. SEQ ID NO.1 shows the cDNA sequence coding for BPLP protein and SEQ ID NO.2 shows the BPLP amino acid sequence.

The BPLP gene products contemplated by the present invention may be for instance the precursor protein or the maturation products of the BPLP precursor protein.

As used herein, a “maturation product of the BPLP” refers to peptides obtained through specific cleavage of the BPLP precursor by natural maturases or prohormone converting enzymes, or related mono or paired basic amino acid-cleaving enzymes such as furin, PC convertases or PACE 4 (Seidah et al., Intramolecular Chaperones and Protein Folding, 1995, 9: 181-203). Prohormone convertases convert an inactive precursor into active peptides and include, e.g., furin, PC convertases or PACE 4. The sequence of the maturation consensus sites preferably follows the following consensus: [H/R/K]—X₃—[R/K]—[R/K] wherein [H/R/K] means that the amino acid is H, R or K, X₃ designates a chain of three amino acids, and [R/K] means that the amino acid is R or K. Prohormone convertases cleave the consensus unit between dibasic residues [R/K]—[R/K]. Prohormone convertases are well known to one skilled in the art and are notably described by Scamuffa et al. (2006 FASEB J. 20(12):1954-63). One of the maturation products of the BPLP contemplated by the present invention is Opiorphin, a pentapeptide having the amino acid sequence QRFSR (SEQ ID NO: 3).

The peptides described in the present invention may be prepared in a conventional manner by peptide synthesis in liquid or solid phase by successive couplings of the different amino acid residues to be incorporated (from the N-terminal end to the C-terminal end in liquid phase, or from the C-terminal end to the N-terminal end in solid phase) wherein the N-terminal ends and the reactive side chains are previously blocked by conventional groups. For solid phase synthesis the technique described by Merrifield may be used in particular. Alternatively, the technique described by Houbenweyl in 1974 may also be used. For more details, reference may be made to WO 98/37100. The peptides according to the present invention may also be obtained using genetic engineering methods.

2—Method of Use of BPLP Gene Products.

According to another aspect of the invention, there is provided a method of use of BPLP gene products. More particularly, the inventors of the present invention provide the use of BPLP gene products (e.g. Opiorphin) as a biological marker in various biological fluids, such as, but not limited to, saliva for patients developing for instance Mouth Burning Syndrome which is a chronic pain syndrome with unknown etiology; plasma and urines for patients suffering for instance of depression and associated social disorders or neuropathic chronic pain; sperm for patients suffering for instance of erectile dysfunction.

This particular aspect of the present invention is advantageously achieved by a method for prognosis, diagnosis or monitoring of a pathological state in a subject. Prognosis or prognostic test refers to the determination or confirmation of a likelihood of a pathological state to arise in a subject. Diagnosis or diagnostic test refers to the determination or confirmation of a pathological state in a subject. Monitoring test refers to the determination or confirmation of the evolution of a pathological state including the efficiency of a treatment, in a subject. It will be understood that the pathological state, the disorder, the disease, or the condition derived from the pathological state refer to any health change relative to a healthy subject.

In a specific embodiment, the method according to the invention aims at determining and/or confirming that a pathological state in a subject is linked with a BPLP gene product.

In a preferred embodiment, sperm is used when the method or use according to the invention aims at prognosis, diagnosis or monitoring a sexual disorder, or determining and/or confirming that a sexual disorder in a subject is linked with a BPLP gene product. Sexual disorders include, e.g., male erectile dysfunction, priapism, hypoactive sexual desire disorder or hyperactive sexual desire disorder.

In another preferred embodiment, cerebrospinal fluid is used when the method or use according to the invention aims at prognosis, diagnosis or monitoring an impaired interpersonal and behavioural disorder, or determining and/or confirming that an impaired interpersonal and behavioural disorder in a subject is linked with a BPLP gene product. Impaired interpersonal and behavioural disorder include, e.g., avoidance disorder, decreased awareness disorder, autistic disorder, arousal disorder, hospitalism, impaired interpersonal functioning and relationship to the external world, schizoid personality disorder, schizophrenia, depressive disorder, major depression syndrome, decreased interest in environment, impaired social activity linked to sexuality, impaired sexual behaviour, including untimely ejaculation and hyperactive sexuality, panic disorder, chest pain and posttraumatic stress disorder, attention deficit (in adults and in children), hyperactivity (in adults and in children), attention-deficit/hyperactivity disorder (ADHD), obsessive-compulsive disorders (OCD) and mood disorders.

In still another preferred embodiment, saliva is used when the method or use according to the invention aims at prognosis, diagnosis or monitoring a Mouth Burning Syndrome or temporomandibular pain disorders.

Plasma, serum and urine are useful for prognosis, diagnosis or monitoring any pathological state described herein, and for determining and/or confirming that pathological state described herein is linked with a BPLP gene product.

A patient or a subject as used herein consists of a vertebrate, e.g. a mammal, such as a human being, regardless of his/her age, sex and general condition. Children and infants are also encompassed except when the studied biological fluid is sperm or milk. The subject who is evaluated or the test subject may be asymptomatic, may be considered likely to develop the disease or condition or may be symptomatic for the disease or condition. Subjects with a suspicion of a target disorder or subjects who have already shown symptoms of the disease or condition or pathological state can also be tested. According to the present invention, the subject is preferably a human being.

The contemplated method of the invention thus comprises the steps of:

a) measuring the quantitative level of a BPLP gene product as defined above in a biological sample obtained from the subject;

b) comparing the quantitative level of the BPLP gene product measured in step a) to a reference value of the BPLP gene product;

wherein a significant higher or lower level of the BPLP gene product compared to the reference value is an indication for the prognosis, diagnosis or evolution of the pathological state.

The method according to the invention may further comprise the step of designing a treatment regimen for said subject. For example, if a significant higher level of a BPLP gene product is found in a subject, a treatment based on the use of a BPLP gene product inhibitor may be designed. The inhibitor may, e.g., inhibit secretion and/or production of the BPLP gene product, or its biological activity. To the contrary, if a significant lower level of a BPLP gene product is found in a subject, a treatment based on the use of a BPLP gene product agonist may be designed. Such agonists include, e.g., the peptides of SEQ ID NO: 3 or 4, and the peptides, peptide derivatives and peptidomimetics described in WO/2009/124948.

For prognosis or diagnosis, the “reference value” is established by statistical analysis of values obtained from a representative panel of healthy subjects. It may depend from the nature of the sample, the age and/or sex of the subject, the neuroendocrine status etc. It may be predetermined to the measure of the quantitative level of a BPLP gene product in a subject.

For monitoring, the “reference value” can be established as described in the above paragraph, or alternatively or additionally be a value obtained from the subject previously tested.

The control subject is either a healthy subject or, when the evolution of the pathological state of a subject needs to be evaluated, the subject may be the subject previously tested.

It will be understood that, as used herein a “biological sample” refers to a fluid from a subject, including serum, plasma, blood, cerebrospinal fluid, urine, milk, tears, sperm, saliva or a tissue extract or a tissue or organ biopsy such as brain, spinal cord, bone tissue, kidney, prostate and gonadal glands, placenta, dental tissue, glandular mucosa of stomach, intestine, salivary gland tissue, mammary glands, for example.

The BPLP-protein maturation product can be extracted from the sample, e.g., by acid-methanol extraction and/or by C18-SepPak cartridge extraction. It may then be purified by reverse phase C18-HPLC chromatography. When the sample is saliva, sperm, tears, or urines, the BPLP-protein maturation product can for example be extracted by acid-methanol extraction. When the sample is plasma or milk, the BPLP-protein maturation product is preferably extracted by C18-SepPak cartridge extraction. When the sample is plasma, the BPLP-protein maturation product can be purified by reverse phase C18-HPLC chromatography.

As used herein, the term “significant” means that the p value is inferior or equal to 0.05. The quantitative level of expression or production of BPLP gene products may be determined for instance by assaying the BPLP mRNA, BPLP precursor protein or its maturation products such as the Opiorphin-pentapeptide. Such assay methods comprise contacting a biological sample with a binding partner capable of selectively interacting with a BPLP gene product present in the sample. The binding partner may be an antibody, which may be polyclonal or monoclonal, or a molecular probe.

Methods for producing antibodies can be easily adapted to produce antibodies useful for the diagnostic, prognostic or monitoring methods according to the invention. For example, the presence or production of the BPLP gene product, can be detected by contacting a biological sample with an antibody that specifically recognizes the BPLP gene product e.g. using standard electrophoretic and liquid or solid immunodiagnostic techniques, including immunoassays such as competition, direct reaction, or sandwich type assays. Such assays include, but are not limited to, Western blots; agglutination tests; enzyme-labelled and mediated immunoassays, such as ELISAs; radioimmunoassay such as those using radioiodinated or tritiated BPLP protein or any of its maturation products; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith. Solid supports which can be used in the practice of the invention include supports such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like. Thus, in one particular embodiment, the presence of bound BPLP gene products from a biological sample can be readily detected using a secondary binding agent comprising another antibody, which can be readily conjugated to a detectable enzyme label, such as horseradish peroxidase, alkaline phosphatase or urease, using methods known to those of skill in the art. An appropriate enzyme substrate is then used to generate a detectable signal, such as a chromogenic or fluorogenic signal for example. In other related embodiments, competitive-type ELISA techniques can be practiced using methods known to those skilled in the art. The above-described assay reagents, including the antibodies, can be provided in kits, with suitable instructions and other necessary reagents, in order to conduct immunoassays as described above. The kit can also contain, depending on the particular immunoassay used, suitable labels and other packaged reagents and materials (i.e. wash buffers and the like). Standard immunoassays, such as those described above, can be conducted using these kits.

In a preferred embodiment, the quantitative level of the BPLP gene product is measured with an immuno-assay preferably comprising a polyclonal anti-QRFSR antibody, such as e.g. a competitive ELISA.

Methods for carrying out competitive ELISA assays are well-known to the skilled in the art. The method comprising the following steps has been found to be especially advantageous and sensitive for detecting Opiorphin:
coating a micro-titration plate with a peptide of SEQ ID NO 4 in which a 6-polyethylene or a 12-polyethylene linker has been introduced between the first and the second residues (e.g. 40 ng of said peptide per 200 μl/wells);
optionally incubating said micro-titration plate, for example over-night at 4° C. under light agitation;
optionally washing said micro-titration plate, for example 2-8 times, preferably 4-5 times;
optionally adding a saturation buffer and washing said micro-titration plate, for example 2-8 times, preferably 4-5 times;
adding a sample susceptible of containing Opiorphin (preferably pre-incubated overnight at 4° C.) in the presence of a rabbit polyclonal anti-QRFSR antibody according to the invention (for example at 1/80 000);
optionally washing said micro-titration plate, for example 2-8 times, preferably 4-5 times;
adding a labelled anti-rabbit antibody at e.g. at 1/3 000 (for example the IgG-HRP antibody from Pierce);
optionally incubating said micro-titration plate, for example for 1 hour at 22° C.;
optionally washing said micro-titration plate, for example 2-8 times, preferably 4-5 times;
adding a chromogenic substrate suitable for detecting the labelled anti-rabbit antibody (e.g. the HRP chromogenic substrate from Pierce);
optionally incubating said micro-titration plate, for example for 30 min at 22° C.;
optionally stopping the reaction (e.g. by adding 4N H2SO4); and
detecting the signal emitted by the chromogenic substrate (e.g. by measure the absorbance at 450 nm wavelength), thereby detecting (and/or quantifying) Opiorphin in said sample susceptible of containing Opiorphin.

The anti-QRFSR antibody can for example be obtained by using the method described in the examples. Such a method of producing a polyclonal antibody specifically binding to the QRFSR-peptide Opiorphin of SEQ ID NO: 3 can comprise the steps of:
a) administering a peptide of SEQ ID NO: 4, conjugated in a covalent manner to a ovalbumin-(CO—NH)— molecule, to a non human animal such as a rabbit; and
b) selecting a polyclonal antibody specifically binding to said QRFSR-peptide.
   The peptide of SEQ ID NO: 4 conjugated in a covalent manner to a ovalbumin-(CO—NH)— molecule can for example correspond to the peptide of SEQ ID NO: 5, in which the ovalbumin-(CO—NH)— molecule is covalently bound to the first residue of the peptide of SEQ ID NO: 4.

The invention also pertains to a polyclonal antibody specifically binding to the QRFSR-peptide Opiorphin of SEQ ID NO: 3 obtainable by such a method (e.g. the 3RBF-SAB antibody).

A subject of the invention is also a method for obtaining a hybridoma that secretes an antibody specifically binding to the QRFSR-peptide Opiorphin of SEQ ID NO: 3, comprising the steps of:
administering a peptide of SEQ ID NO: 4, conjugated in a covalent manner to a ovalbumin-(CO—NH)— molecule, to a non human animal such as a rabbit;
removing immunoglobulin-secreting lymphocytes from this animal;
fusing the lymphocytes with myeloma cells so as to obtain at least one immunoglobulin-secreting hybridoma;
selecting a hybridoma that secretes said antibody specifically binding to the QRFSR-peptide Opiorphin of SEQ ID NO: 3; and
optionally purifying said antibody.
   These steps correspond to those of the conventional technique for obtaining hybridomas, the principle of which is described in Köhler and Milstein, (1975 Nature 256: 495-497).

A subject of the invention is also such a hybridoma and the antibody secreted by said hybridoma.

The quantitative level of BPLP gene products may also be measured with the mRNA. An oligonucleotide hybridizing specifically with the mRNA expressed by the BPLP gene or a fragment thereof may be used. The person skilled in the art knows how to prepare such an oligonucleotide, once the BPLP gene sequence or the cDNA sequence is known. For instance, the hybridization may be obtained by using a solution containing: 0.5M sodium phosphate, pH 7.2; 7% sodium dodecylsulfate (SDS); 1 mM EDTA; 1% bovine serum albumin; and sonicated salmon sperm DNA: 100 mg/mL (U.S. Pat. No. 6,916,607, Rosinski-Chupin et al.).

Firstly, the level of the BPLP gene product is measured in a representative panel of healthy subjects, or in the subject for which the evolution of the pathological state is monitored, to obtain the “reference value” of the BPLP gene product.

Secondly, the level of the BPLP gene product is measured in a subject.

Thirdly, the levels are compared. If they are significantly different (p≦0.05), either lower or higher, it may be useful for the prognosis, diagnosis or the monitoring of a particular pathological state.

As described in the examples, under the conditions of oral mucosal stimulation by citric acid, salivary secretion of Opiorphin was significantly increased in men: 738±146 compared to basal secretion: 59±17 ng/ml, p<0.001, n=6-10; and in women: 247±68 vs basal: 61±23 ng/ml, p<0.01, n=10-13.

In a subject with a pathological state, such as, but not limited to, hydromineral imbalance, hyper- or hypo-algesic syndromes, depressive states and socio-relational behavioral imbalances, the level of BPLP gene products will be different than in a control subject and will be an indication of that pathological state. The pathological states that can be prognosed, diagnosed or monitored in the frame of the present invention will be further described herebelow.

The method of use of BPLP gene products can be used as a marker for evaluation of efficacy of treatments.

In order to determine the evolution of a condition and/or to determine the efficiency of a treatment, it may be very useful to test a subject for the expression of a BPLP gene product and to monitor the effect of a drug or the spreading of the condition, by testing him/her a second time, e.g. a few days, weeks, or months later. In that case the results of the second test can be compared with the results of the first test, and in general also with the results obtained with a “healthy” subject. The “control subject” then refers either to the same test subject or to a “healthy subject”.

A kit for the prognostic, diagnostic or monitoring test using a BPLP gene product as a biomarker is also provided. The kit may comprise a binding agent (e.g. an antibody or a molecular probe) for specifically recognizing BPLP gene products and reagents to detect the binding of the agent with BPLP gene products. Control samples (positive and/or negative) can also be included.

3—Pathological States

BPLP gene products which modulate the activity of membrane metallo-ectopeptidases such as NEP and APN, are useful for prognosing, diagnosing or monitoring a wide variety of pathological states. For example, PCT/IB2005/000700 teaches numerous diseases in which BPLP gene products play a role.

Therefore, the methods and uses according to the invention are useful for prognosing, diagnosing or monitoring pain, especially acute and chronic pain, visceral inflammatory and neuropathic pain. In particular, the methods and uses according to the invention are useful for prognosing, diagnosing or monitoring hyperalgesia, including fibromyalgia, chronic back pain, temporomandibular pain disorders, stomatodynia and visceral pain hypersensivity.

The prognosis, diagnosis or monitoring of any hydro-mineral imbalance is also an aim of the invention. Among target disorders one may cite bone, teeth, kidney, parathyroid, pancreas, intestine, stomach mucosa, prostate, and salivary gland disorders that are caused by hydro-mineral imbalance. In particular, the disorder may be selected from the group consisting of hyper or hypo-parathyroidism, osteoporosis, osteopenia, hypophophatemia, pancreatitis, submandibular gland lithiasis, nephrolithiasis and osteodystrophy.

The prognosis, diagnosis or monitoring of impaired interpersonal and behavioural disorders is of further interest. Various such mental disorders are described in WO 02/051434. In particular the invention is drawn at any pathological state selected from the group consisting of avoidance disorder, decreased awareness disorder, autistic disorder, attention deficit, hyperactivity disorder, arousal disorder, hospitalism, impaired interpersonal functioning and relationship to the external world, schizoid personality disorder, schizophrenia, depressive disorder, major depression syndrome, decreased interest in environment, impaired social activity linked to sexuality, impaired sexual behaviour, including untimely ejaculation and hyperactive sexuality, panic disorder, chest pain and posttraumatic stress disorder.

Pathological states according to the invention also include neurodegenerative diseases. The term “neurodegenerative disease” refers to a disease or disorder of the nervous system, particularly involving the brain, that manifests with symptoms characteristic of brain or nerve dysfunction, e.g., short-term or long-term memory lapse or defects, dementia, cognition defects, balance and coordination problems, and emotional and behavioral deficiencies.

The present invention is more particularly concerned with neurodegenerative diseases that are associated with amyloidosis. Such diseases are “associated with amyloidosis” when histopathological (biopsy) samples of brain tissue from subjects who demonstrate such symptoms would reveal amyloid plaque formation. As biopsy samples from brain, especially human brain, are obtained with great difficulty from living subjects or might not be available at all, often the association of a symptom or symptoms of neurodegenerative disease with amyloidosis is based on criteria other than the presence of amyloid deposits, such as plaques or fibrils, in a biopsy sample.

In a specific embodiment, according to the present invention the neurodegenerative disease is Alzheimer's disease (AD). Other such diseases known in the art and within the scope of the present invention include, but are not limited to, sporadic cerebral amyloid angiopathy, hereditary cerebral amyloid angiopathy, Down's syndrome, Parkinson-dementia of Guam, and age-related asymptomatic amyloid angiopathy.

In addition, PCT/EP2009/056390 teaches that opiorphin has a psychostimulant activity. Therefore, the methods and uses according to the invention can be used for prognosing, diagnosing or monitoring a pathological state selected from the group consisting of narcolepsy, hypersomnia, vigilance drop, attention deficit (in adults and in children), hyperactivity (in adults and in children), attention-deficit/hyperactivity disorder (ADHD), obsessive-compulsive disorders (OCD), and mood disorders such as depressive conditions and depression (“Major Depressive Disorder”), the latter including primary depression (“Major Depressive Disorder, Single Episode”) and resistant depression (“Major Depressive Disorder, Recurrent”), bipolar disease (of type I and/or of type II), dysthymic disorder and cyclothymic disorder.

The present invention will be more readily understood by referring to the following examples. These examples are illustrative of the wide range of applicability of the present invention and are not intended to limit its scope. Modifications and variations can be made therein without departing from the spirit and scope of the invention. Although any methods and materials similar or equivalent to those described herein can be used in the practice for testing of the present invention, the preferred methods and materials are described.

### EXAMPLES

### Material and Methods

A. Sampling, Conservation and Transport of Biological Fluids

The protocol was the following one:
Biologic samples were taken and collected (i.e., 2-5 ml of saliva, 30 ml of blood, 20 ml of urine by subject) into previously cooled polypropylene tubes (4, 15 or 50 ml) containing a mixture of peptidase inhibitors: Pefabloc 0.4 mM, Aprotinin 1000 KIU/ml, EDTA 1 mM, bestatine 150 μM, leupeptin 1 μM, pepstatin 1 μM (Rougeot and al. Am J Physiol, on 1997). For the sperm the peptidase inhibitors were limited (according to previous validation tests) to Aprotinin and Pefabloc in absence of the anti-coagulant (EDTA).
They were centrifuged for 15 min at 4000×g and 4° C. and the supernatants were collected (i.e., plasma fraction for the blood);
All samples were immediately stored at −80° C.; and
They were transported in dry ice container to the laboratory.

B. Development of the Quantitative Immuno-Assay for Opiorphin

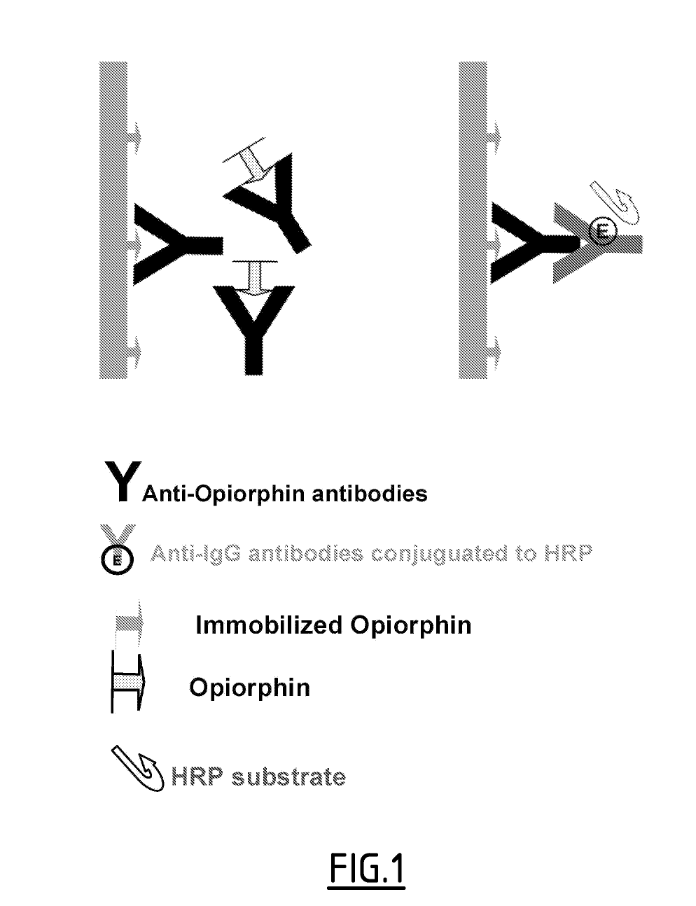

Considering the small size of the Opiorphin (650 Da), an immuno-assay of competitive type was applicable to the quantification of the peptide: i.e., competition between the free antigen to be measured, the Opiorphin QRFSR-peptide (contained in the sample or in the known concentrations of the reference-peptide) and the antigen coated to the support of the 96 well-micro-titration plate (in constant and limited quantity) towards the “anti-QRFSR-peptide” antibodies (in constant and limited quantity) (FIG. 1). Y represents the antibody directed against Sialorphin.

On the other hand, for the same reason, the production of the polyclonal “anti-QRFSR” antibodies in the animal (rabbit) required the injection of the antigen, to improve the immunogenicity of the QRFSR-peptide, conjugated in a covalent manner to a carrier molecule (generally a protein from various species excepted human). The immunogen used was ovalbumin-(CO—NH)-YQRFSR (also called OVA-(CO—NH)-YQRFSR) (SEQ ID NO: 5).

The specific reaction was revealed by using an universal system supplied by Pierce, i.e., purified immunoglobulin antibodies (IgG-IgM) against rabbit immunoglobulin

(IgG) conjugated to the horseradish peroxydase enzyme (HRP) followed by final addition of the chromogenic TMB specific substrate (tetramethylbenzidine).

B.1 Format of the Competitive ELISA Immuno-Assay for Opiorphin:—Anti-YQRFSF Antibody and—Immobilization of the Y-(PE)₁₂-QRFSR Peptide

The peptide was coated on the plastic support of the micro-titration plate (96-well-ImmunoPlate, Nunc). Different QRFSR-peptide derivatives were tested:
The Y-QRFSR peptide (SEQ ID NO: 4): the presence of the N-terminal tyrosine residue increases the hydrophobic character of the Opiorphin-peptide, and so as to facilitate its passive adsorption to the plastic support;
The Y-(PE)₆-QRFSR and Y-(PE)₁₂-QRFSR peptides (SEQ ID NO: 6): the presence of the 6-polyethylene or 12-polyethylene linker introduced between the tyrosine residue and the QFRSR-peptide sequence facilitates the accessibility of the immobilized peptide to the antibody by rending it more flexible and farther from the coated-support as well as more hydrophobic. As expected, the more the sequence QFRSR is taken away from the support, the more its recognition by the antibody is facilitated. The Opiorphin derivative selected for the plate coating was the Y-(PE)12-QRFSR peptide.

The specific polyclonal anti-QRFSR antibody was generated after administration of the OVA-CO—NH-YQRFSR immunogen to 2 rabbits and a precise follow-up of the specific immune response. The polyclonal antibody selected on the basis of its affinity for Opiorphin was referred as 3RBF-SAB.

The optimized assay conditions were the following ones:

Coating buffer: potassium phosphate buffer at 100 mM and pH 7.1.

Saturating buffer: Tris buffer at 20 mM pH 7.5+150 mM NaCl+0.1% Tween 20+0.5% gelatin.

1st Incubation buffer (anti-Opiorphin antibody+sample or reference-peptide): Tris buffer at 200 mM and pH7.5+150 mM NaCl+0.1% Tween 20+0.1% bovine serum albumin (BSA).

2nd Incubation buffer (anti-rabbit IgG antibody conjugated to HRP): Tris buffer at 20 mM and pH 7.5+150 mM NaCl+0.1% Tween 20+0.1% BSA.

Washing buffer: pure water+0.1% Tween 20.

The following Table 1 describes in more details the steps of the competitive ELISA immunoassay.

TABLE 1

| | |
|---|---|
| Coating | 40 ng of Y-(PE)₁₂-QRFSR peptide per 200 μl/wells (96-wells micro- |
| | titration plates, Immuno-Plate, Nunc) |
| | Incubation over-night (O/N) at +4° C. under light agitation |
| | Washing 5-times |
| Saturation | 250 μl saturation buffer - Incubation 1 h. at 22° C. |
| | Washing 5-6 times |
| 1^{st} | 50 μl of diluted samples or known concentrations of the reference |
| Immunological | Opiorphin-peptide (from 2 ng/ml to 500 ng/ml) pre-incubated O/N at |
| Reaction | 4° C. in the presence of 50 μl of anti-Opiorphin antibody at 1/80 000 |
| | Incubation on the coated micro-titration plates 1 h. 30 min. at 4° C. |
| | Washing 5-6 times |
| 2^{nd} | 100 μl of the conjugate anti-rabbit IgG-HRP (Pierce) at 1/3 000 |
| Immunological | Incubation 1 h. at 22° C. |
| Reaction & | Washing 5-6 times |
| Revelation | 100 μl of the HRP chromogenic substrate (Step Ultra TMB-ELISA, |
| | Pierce): Incubation 30 min at 22° C. |
| | Stop the reaction according to the manufacture conditions (100 μl 4N |
| | H₂SO₄) Measure the absorbance at 450 nm wavelength |

8.2. Characteristics of the Competitive ELISA Immuno-Assay for Opiorphin

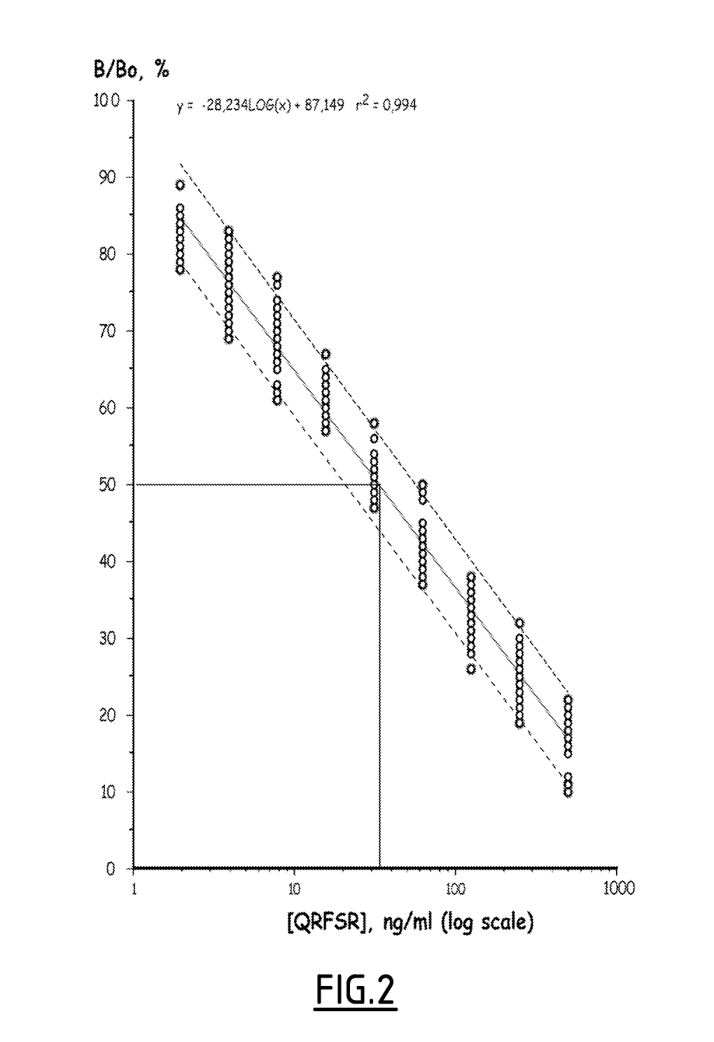

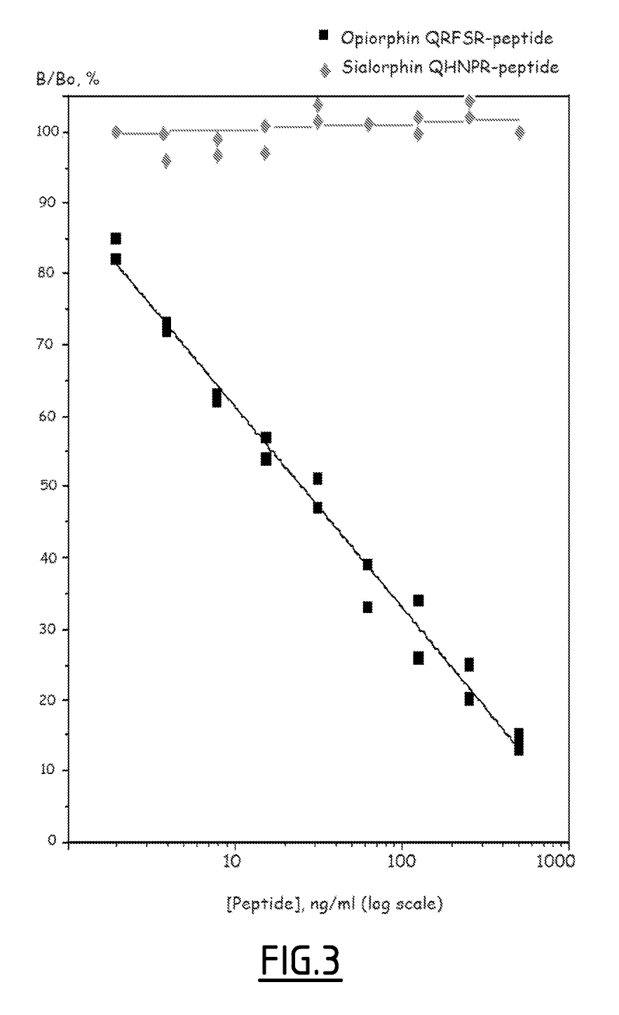

As illustrated in FIG. 2, the competitive-ELISA immuno-assay for Opiorphin, using the anti-YQRFSR antibody 3RBF-SAB, allowed to perform a reproducible and sensitive assay: the IC50 was 30±9 ng/ml (SD, n=18 standard curves) (3 ng or 5 pmol/assay) and detection limit was 2 ng/ml of Opiorphin QRFSR-peptide (200 pg or 0.3 pmol/assay). Inter-assay variability was less than 10%. In addition, the immuno-assay was highly specific for Opiorphin as the functional related Sialorphin QHNPR-peptide (lozenge) was not recognized by the antibody even at 500 ng/ml final concentration (FIG. 3).

C. Extraction of Opiorphin from the Human Biological Samples

Briefly, after de-freezing at +4° C., the biological samples were treated according to the following conditions:

C.1. Acid-Methanol Extraction: 1 Volume Sample for 4 Volumes of 0.1% Trifluoroacetic Acid (TFA) in Methanol at +4° C.

This first step realized the precipitation and the elimination of high molecular weight proteins (i.e., peptidases) which are denatured in acid-methanolic conditions; the soluble low molecular weight molecules (i.e., Opiorphin) in the methanol phase was separated from the precipitate by centrifugation at 4700 rpm during 30 min at +4° C. and lyophilized at −110° C. during 48 h.
In the case of sperms and plasmas, samples were previously acidified with HCl at 0.1N final concentration (dissociation of potential ionic interactions between Opiorphin and binding components). In addition, in the case of sperms, samples were previously treated with EDTA at 1 mM final concentration (dissociation of Opiorphin from associated cationic metal, i.e., Zn++ concentrated in seminal fluid at about 1.5 mM).

The saliva, tears and milk, dried-extracts were reconstituted in 1 volume of H2O-HPLC (corresponding to the initial volume of samples) centrifuged (4700 rpm, 30 min at +4° C.) and the supernatants were conserved at −80° C. until to be analyzed by ELISA for their Opiorphin content.
*In the case of sperms, dried-extracts were reconstituted in 0.2 volume of H2O-HPLC compared to the initial volume of samples (final concentration at 5×).
*In the case of urines, dried-extracts were reconstituted in 0.4 volume of H2O-HPLC compared to the initial volume of samples (final concentration at 2.5×)

C.2. C18-SepPak Cartridge Extraction (Waters)

The acidified samples (HCl, 0.1N final concentration) were applied to the pre-activated C18-SepPak cartridges. After washing with H2O-0.1% TFA (5 ml), the components were eluted according to a multi-step gradient of: 5%-20%-40%-60% and 100% methanol-0.1% TFA (5 ml each). The successive fractions were collected at 4° C. and then lyophilized at −110° C. during 48 h.
*This extraction-purification step has been critical in the case of plasmas and milk.

C.3. Reverse Phase C18-HPLC Chromatography

The extracts obtained during the procedures described above were applied to the top of the C18/RP-HPLC column (Luna 5μ Phenomenex-USA or ACE 3μ AIT-France (150×4.5 mm)) under TFA 0.1%-H2O solvent conditions.

The various components were eluted and isolated according to their hydrophobic characteristic, in a 30-min linear gradient: from 0% to 80% acetonitrile, and at a 1 ml/min flow rate (Surveyor HPLC system, Thermo-scientific). Each fraction (1 ml) was collected and lyophilized as before and tested for their Opiorphin content by ELISA.
*This HPLC purification step has been critical in the case of plasmas.

### Results

Opiorphin Concentration Profile in Human Biological Fluids

### Example 1

### Quantification of Opiorphin in the Saliva from Healthy Human Volunteers

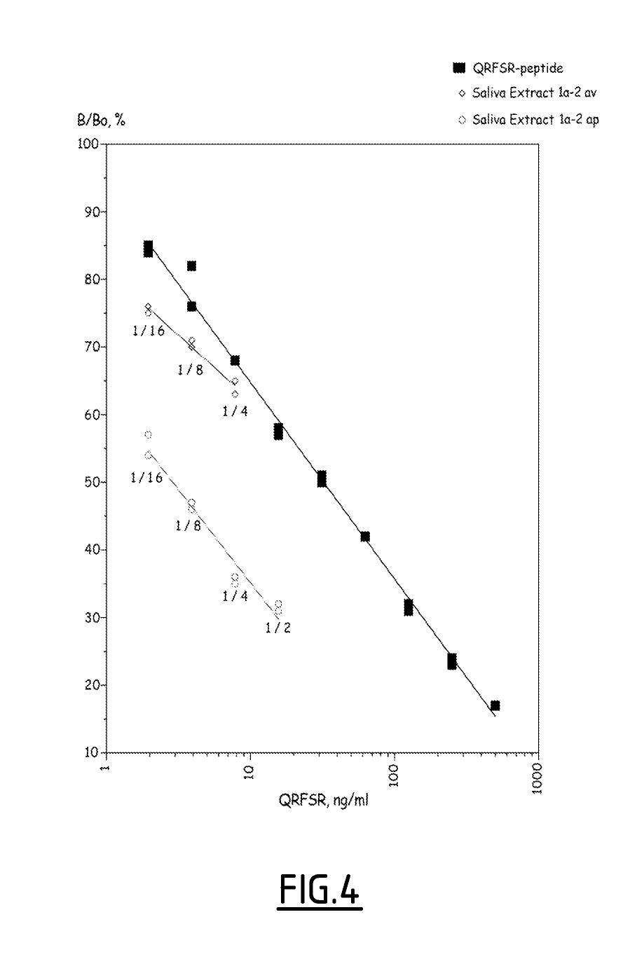

A representative Opiorphin competitive-ELISA assay is shown in FIG. 4; the immuno-reactive curve obtained from successive dilutions of the 1 a-2 sample (open circle) was parallel to the dose-response curve corresponding to the reference QRFSR-peptide (black square, concentrations in abscise axis is in log scale). This assesses that the natural Opiorphin-peptide contained in the saliva extract was recognized by the antibody with the same affinity as the synthetic pure peptide.

In saliva samples collected from healthy adult males, Opiorphin was detected in saliva under basal conditions (open lozenge, FIG. 4) as well as but more abundantly under stimulated conditions of salivary secretions (drop of diluted lemon applied to the mouth floor)(open circle).

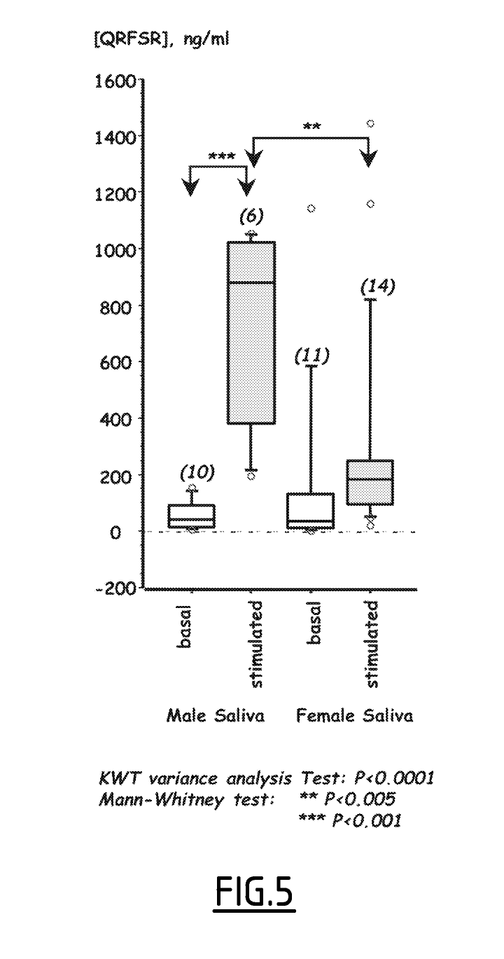

The statistical analysis of altogether human salivary samples collected from healthy men, revealed that the median concentration of Opiorphin is 50 ng/ml (n=10) in basal conditions, while under conditions of chemical stimulation of salivary secretions, it is evaluated at 870 ng/ml (n=6). So salivary secretion of Opiorphin in men was significantly increased under the conditions of oral mucosal stimulation by citric acid (p<0.001, Mann Whitney U-Test, FIG. 5).

For the human saliva samples collected from healthy women of similar age, the median concentration of Opiorphin was evaluated at 57 ng/ml in basal conditions (n=11), and at 141 ng/ml in stimulated conditions (n=14). Given the important heterogeneity in Opiorphin salivary levels among women, the difference between the basal and stimulated conditions, was not significant. However, under the chemical stimulated conditions of salivary secretions, Opiorphin levels in male saliva were significantly higher than those of female saliva (p<0.005 Mann Whitney U-Test, FIG. 5).

It is interesting to note that the rate of Sialorphin salivary secretion in male rats under stimulated conditions of the sympathetic nervous system innervating the salivary glands (injection of Noradrenaline-Pilocarpine) was similar (1 μg/ml) than that achieved for Opiorphin in men under conditions of chemical stimulation of the mouth nerve endings. On the other hand, as for the secretion rate of salivary Opiorphin in humans, that of salivary Sialorphin in male rats was also significantly higher than that of female rats (Rougeot et al, Eur. Biochem J. 1995).

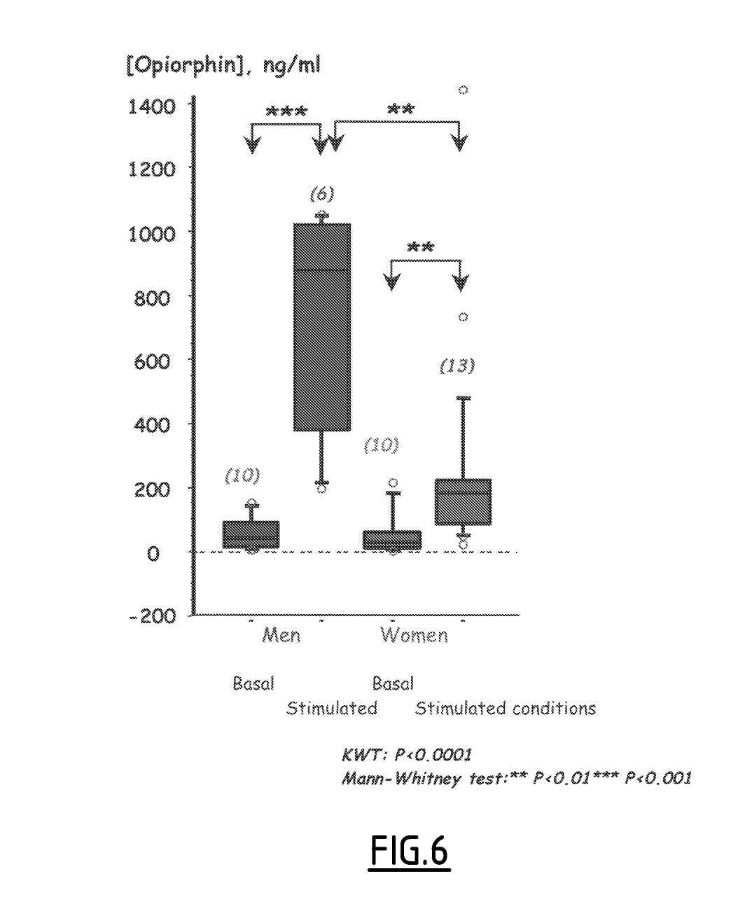

Strikingly, the important heterogeneity observed in the level of salivary Opiorphin of healthy volunteer women was associated with the fact that one of them had (open circle, FIG. 5) a high salivary concentration of Opiorphin both in basal (1072 ng/ml) and in stimulated conditions (1637 ng/ml). The clinical data associated with this sample revealed that the woman was treated with thyroid hormones. Consequently, this sample was excluded from the final statistical analyses presented in FIG. 6.

TABLE 2
Results obtained in healthy male and female saliva.

| | Mean ± Standard Error | |
|---|---|---|
| | of Mean (SEM) | Number |
| | ng Opiorphin/ml saliva | of samples |
| Healthy men | | |
| Basal condition | 59 ± 17 | 10 |
| Stimulated condition | 738 ± 146 | 6 |
| Healthy women | | |
| Basal condition | 61 ± 23 | 10 |
| Stimulated condition | 247 ± 68 | 13 |

Finally, statistical analyses showed that the salivary secretion of Opiorphin in women is significantly increased under the conditions of oral mucosal stimulation by citric acid (p<0.01, Mann Whitney U-Test, FIG. 6) compared to basal conditions.

### Example 2

### Quantification of Opiorphin in Plasma from Healthy Human Volunteers

A simple acid-methanol extraction procedure (as for saliva) followed by a 5-fold concentration of the dried-extract was insufficient to allow the detection of Opiorphin in plasma, mainly because of the presence of plasmatic components interfering with the ELISA immuno-assay. Thus, the following protocol was applied:

After acidification of the human plasma (HCl, 0.1 N final concentration), the sample was purified on a C18 cartridge-SepPak as described in the chapter “C18-SepPak cartridge Extraction”. Recovery of the marker Opiorphin-peptide (QR [3H]FSR) in fractions eluted with 20% and 40% methanol, was 68%±2%, n=4. Fractions were lyophilized, reconstituted in 250 μl H2O-HPLC (40-fold concentration compared to the 10 ml initial plasma volume) and subjected to C18RP-HPLC chromatography according to the procedure described in chapter “Reverse phase C18-HPLC Chromatography”. The retention time of the reference Opiorphin-peptide in the chromatographic system (ACE column) was 20±0.2 min, n=4. The HPLC fractions, from 15 to 26 min/ml, corresponding to the samples were collected, lyophilized, reconstituted in 120 μl H2O-HPLC and there Opiorphin content was determined by ELISA. Thus, the final plasmatic samples were concentrated 53-fold compared to the initial sample volume corresponding to 10 ml of human plasma.

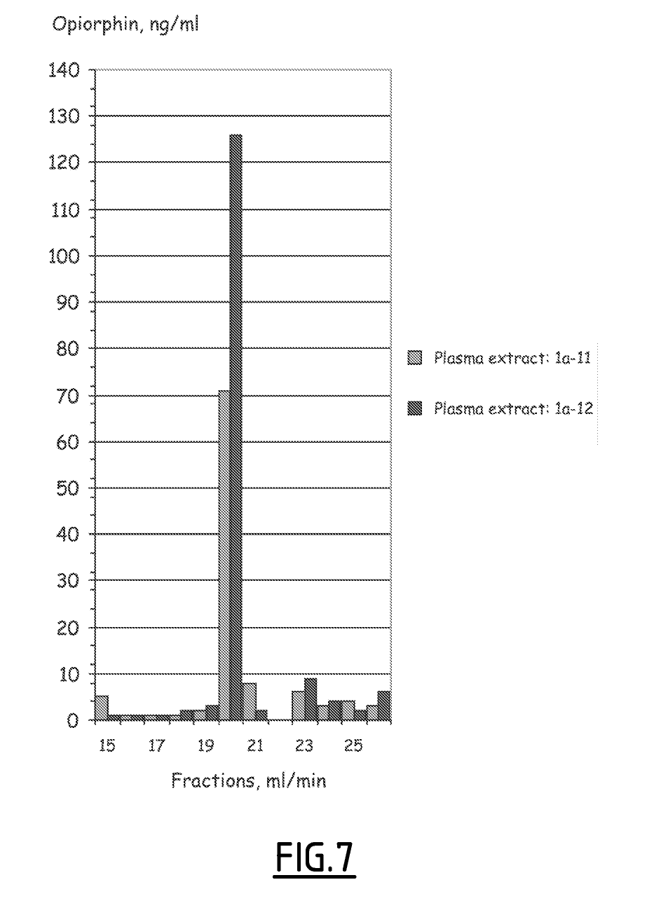

As shown in FIG. 7, HPLC elution profile of the human plasma extracts, 1a-11 and 1a-12 demonstrated that Opiorphin is detected at 20 min retention time, which exactly coincide to that of the reference Opiorphin-peptide. This validation step allowed establishing the plasma level of Opiorphin for 4 human plasmas.

TABLE 3
Results obtained in healthy male and female plasma.

| | Mean ± SEM | | Mean ± SEM |
|---|---|---|---|
| | ng Opiorphin/ml | | ng Opiorphin/ml |
| | concentrated | Number | initial plasma |
| | extract sample | of determinations | volume |
| Healthy men | | | |
| 1a-11 | 73 ± 11 | 9 | 1.4 ± 0.2 |
| 1a-12 | 142 ± 33 | 9 | 2.7 ± 0.6 |
| Healthy women | | | |
| 1b-3 | 34 ± 3 | 4 | 0.6 ± 0.1 |
| 1b-15 | 79 ± 13 | 4 | 1.5 ± 0.2 |

Interestingly the physiological range of circulating Sialorphin in conscious adult male rats that was established at 1-7 ng/ml (Rougeot and al. Am J Physiol, 1997) is similar to the plasma concentration of Opiorphin in humans established at about 0.6-2.7 ng/ml, for 4 samples.

### Example 3

### Quantification of Opiorphin in the Seminal Fluid from Human Volunteers

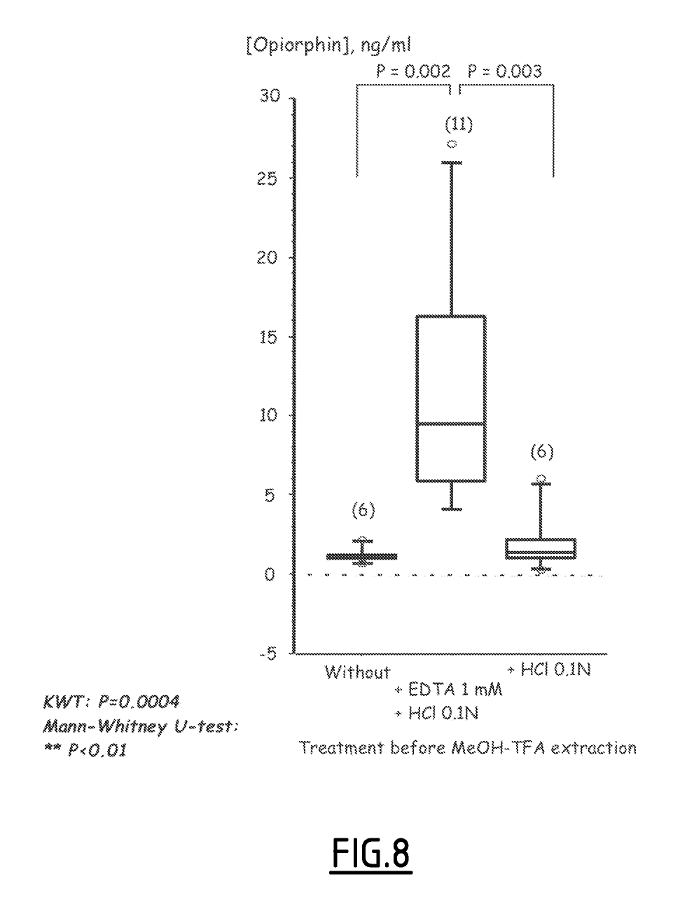

As shown on FIG. 8, previous treatment of human sperm samples by EDTA (1 mM final concentration) before acid-methanol extraction constituted a crucial step for Opiorphin recognition by the anti-Opiorphin antibody. This suggests that Opiorphin in seminal fluid is associated in a molecular complex involving a cation mineral element, i.e., Zn++ ion contained in the sperm (mM concentration range) and constituting a biochemical prostate marker. Recovery of the marker Opiorphin-peptide added to one sample before acid-methanol extraction was 70%.

TABLE 4
Results obtained in male seminal fluid.

| | Mean ± SEM | | Mean ± SEM |
|---|---|---|---|
| | ng Opiorphin/ml | Number | ng Opiorphin/ |
| | sperm | of samples | ejaculation |
| Healthy volunteers | 12.9 ± 1.8 | 11 | 43.6 ± 10.2 |
| Volunteers with | 4.9 ± 1.0 | 4 | 6.4 ± 1.6 |
| congenital bilateral | | | |
| agenesis | | | |

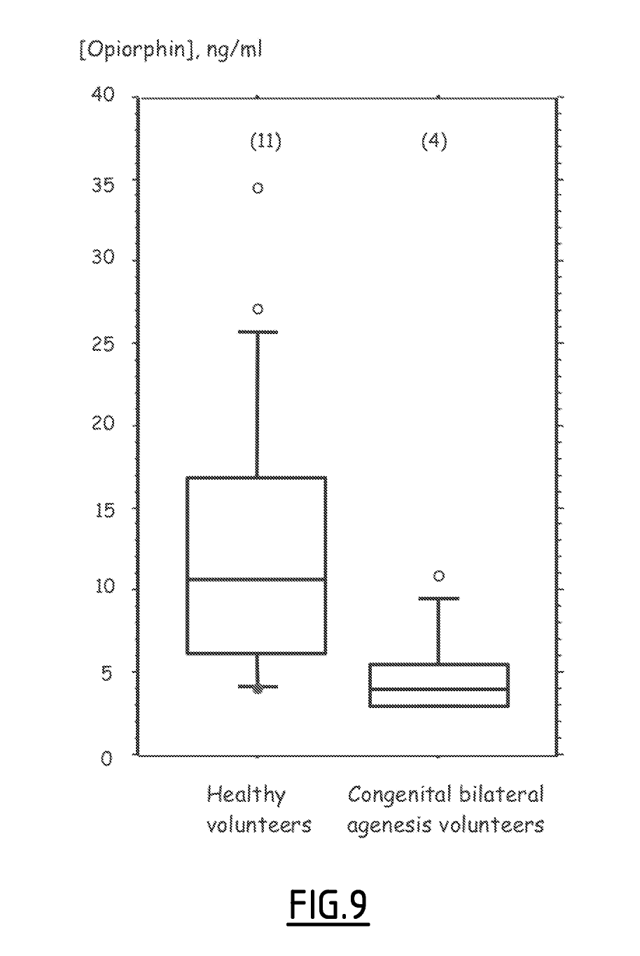

According to these extraction conditions, the physiological concentration range of Opiorphin in human seminal fluid was established at 12.9±1.8 ng/ml or 43.6±10.2 (SEM, n=11) ng/ejaculate volume (FIG. 9). Interestingly, Opiorphin was also detected in sperm of patients with congenital bilateral agenesis, which is associated with anomalies of the vas deferens and seminal vesicle (inducing infertility although testicular spermatogenesis is intact). Although, their Opiorphin levels (4.9±1.0 ng/ml, n=4) were significantly lower than those of healthy male sperms, the presence of Opiorphin in sperm of these patients suggest that secretion of Opiorphin in the seminal fluid originates for the most part from prostate glands.

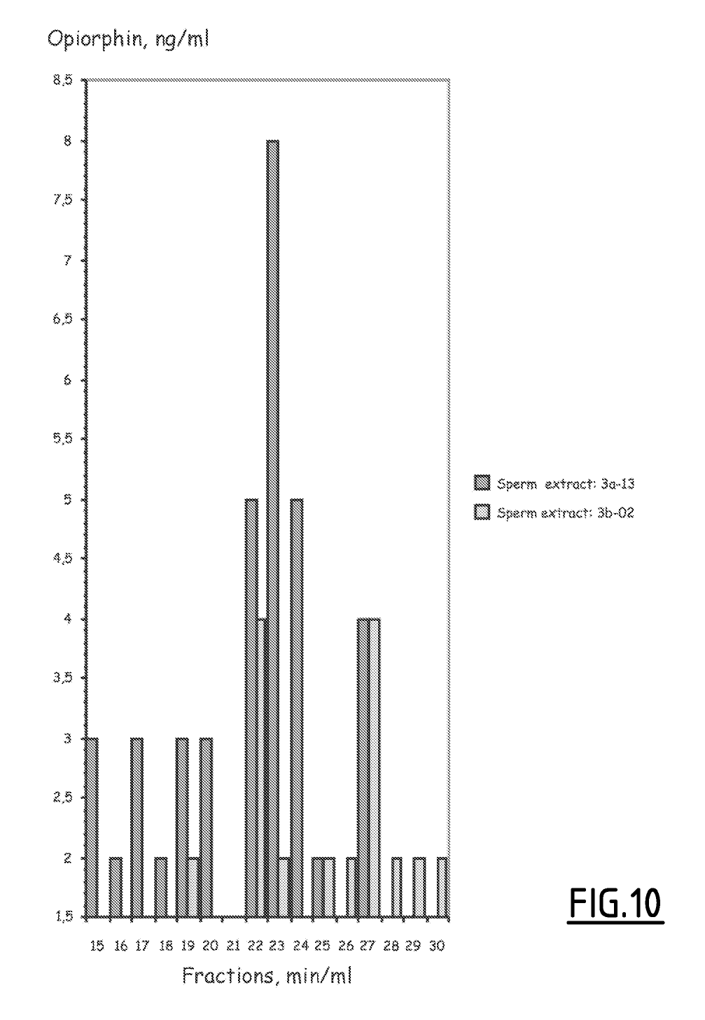

In the RP-HPLC chromatographic system using a Luna column, the retention time of the reference Opiorphin-peptide was 22-23 min. Under these chromatographic conditions, the fractionation and ELISA analysis of the sperm extracts, 3a-13 and 3b-02 (FIG. 10) revealed that the immuno-reactive seminal Opiorphin is primarily eluted at 22-23 min retention time, which well-corresponded to the retention time of reference Opiorphin-peptide. Thus, the authentic presence of Opiorphin in human seminal fluid was validated.

### Example 4

### Quantification of Opiorphin in the Urine from Human Volunteers

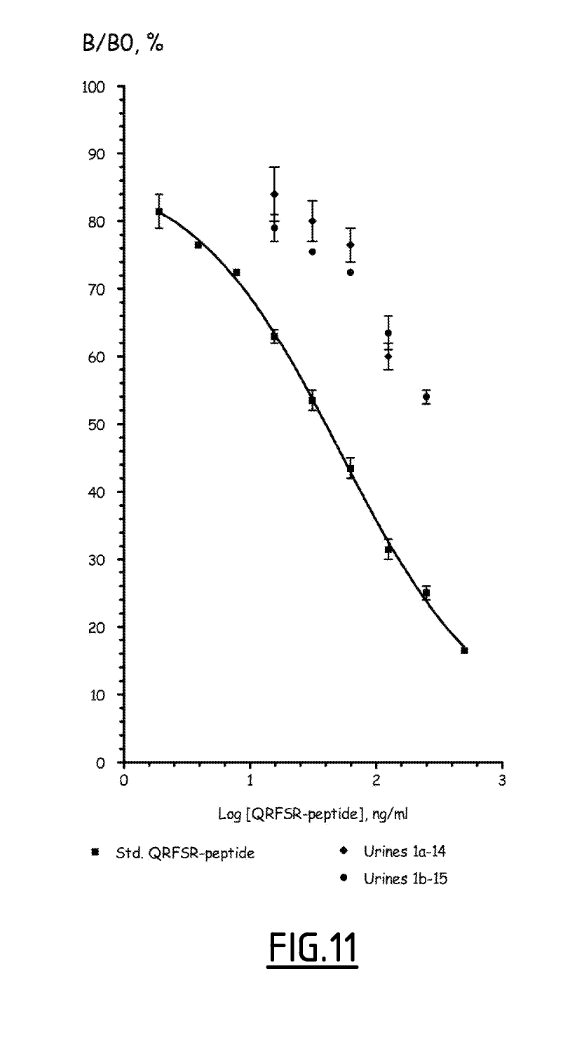

The representative Opiorphin competitive-ELISA assay for the urine samples, 1b-15 and 1a-14 is shown on FIG. 11; the immuno-reactivity obtained from successive dilutions of the urine extracts (black circle and lozenge) was parallel to the dose-response curve corresponding to the reference QRFSR-peptide (black square). This indicates that the natural Opiorphin-peptide contained in these human urines was recognized by the antibody with the same affinity as the synthetic pure peptide.

The percentage of the mean recovery of immunoreactive Opiorphin after incubation of known concentration of synthetic peptide in the presence of urine extract was 100±19% confirming the absence of interfering urine components in the ELISA test.

Otherwise, the RP-HPLC fractionation and ELISA analysis of the urine extract, 3a-14, showed that immuno-reactive Opiorphin is detected at −21-22 min retention time coinciding with the retention time of reference Opiorphin-peptide, but also at −28-29 min retention time as a major immuno-reactive peak. This suggests that Opiorphin in urine fluid is associated to form a more hydrophobic molecular complex. Interestingly, the same kind of HPLC profile was obtained with the sperm extracts in absence of EDTA.

TABLE 5
Results obtained in healthy male and female urine.

| | Mean ± SEM | Number |
|---|---|---|
| | ng Opiorphin/ml Urine | of determinations |
| Healthy men | 20 ± 4 | 4 in duplicate |
| volunteers: 1a-14 | | |
| Healthy women | 14 ± 2 | 5 in duplicate |
| volunteers: 1b-15 | | |

The physiological concentration range of Opiorphin in human urine fluid was established at about 14-20 ng/ml, for 2 samples.

### Example 5

### Quantification of Opiorphin in the Tears of Human Volunteers

TABLE 6
Results obtained in healthy male and female tears.

| | Mean ± SEM | Number |
|---|---|---|
| | ng Opiorphin/ml tears | of samples |
| Healthy men | 49 ± 25 | 9 |
| volunteers | (≦2 ng/ml) | (6) |
| Healthy women | 317 ± 79 | 14 |
| volunteers | (≦2 ng/ml) | (1) |

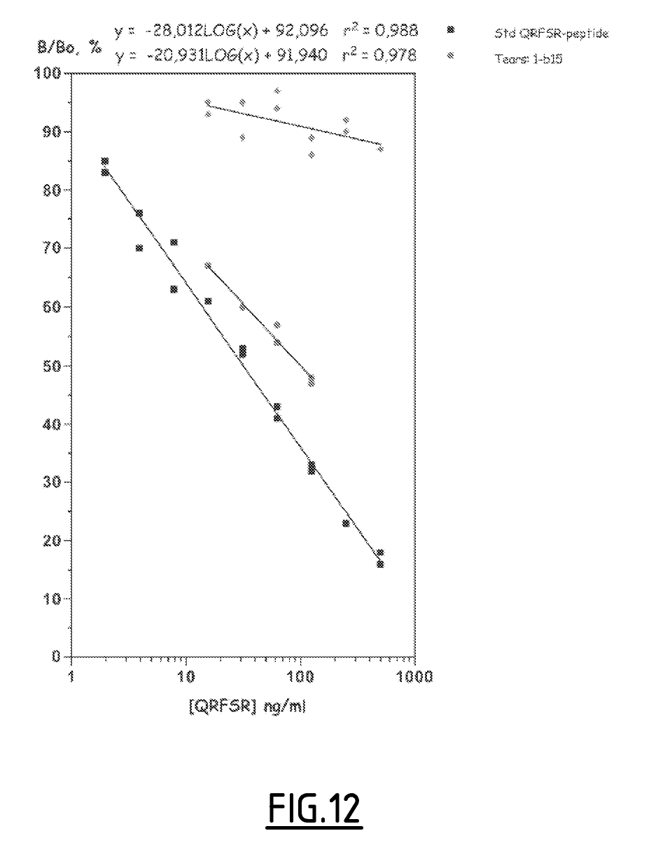

The representative Opiorphin competitive-ELISA assay for the tear samples, 1b-15 and 1a-10 is shown in FIG. 12; the immuno-reactive curve obtained from successive dilutions of the 1b-15 tear extract (circle) was parallel to the dose-response curve corresponding to the reference QRFSR-peptide (black square, concentrations in abscise axis is in log scale). This assesses that the natural Opiorphin-peptide contained in the female tears was recognized by the antibody with the same affinity as the synthetic pure peptide. The Opiorphin levels of male tears (i.e., 1a-10, FIG. 12) were for the majority inferior to the detection limit (6 cases out of 9).

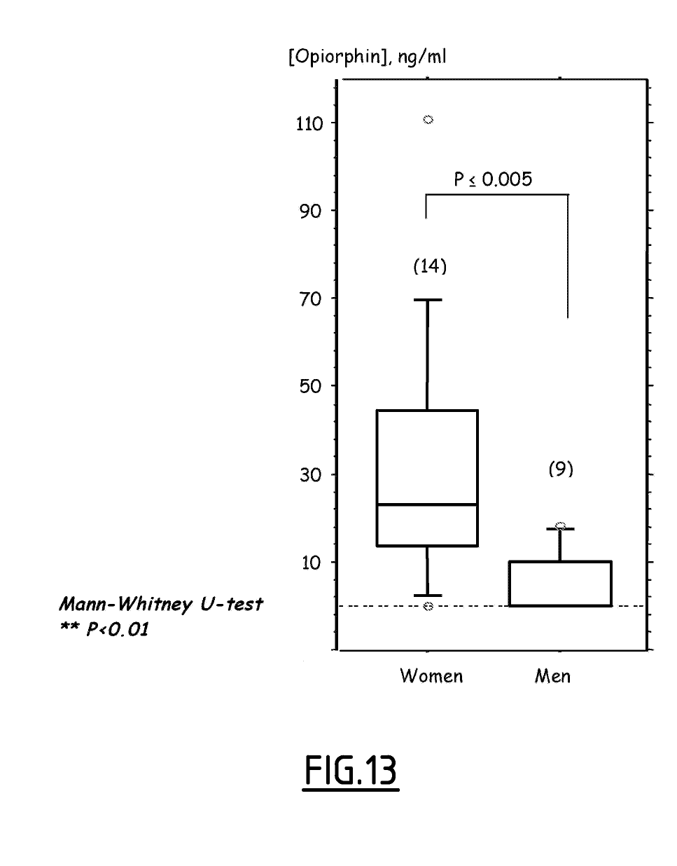

The statistical analysis of altogether human tear samples collected from healthy women, showed that the mean concentration of Opiorphin was 317±79 ng/ml (n=14), while that of healthy men was evaluated at 49±25 ng/ml (n=9). Although an important heterogeneity was observed in the level of Opiorphin in tears, it was significantly increased in women compared to men (p<0.01, Mann Whitney U-Test, FIG. 13).

The RP-HPLC fractionation and ELISA analysis of the tear extracts, 1b-04/1b-06/1b-15, showed that immuno-reactive Opiorphin is detected at −20 min retention time, but also at −26 retention time and −29 min retention time as major immuno-reactive peak.

### Example 6

### Quantification of Opiorphin in the Human Milk

An important Opiorphin immunoreactivity was found in milk samples collected from seven female volunteers, from two to five week post-delivery. However for the major part of milk extracts (under methanol extraction conditions), the immuno-reactive curve obtained from successive dilutions was not parallel to the dose-response curve of the standard QRFSR-peptide, indicating the presence of milk components interfering with the ELISA immuno-assay. In addition, the RP-HPLC fractionation and ELISA analysis of the milk extract, 2-06, showed that immuno-reactive Opiorphin is detected at −21 min retention time, but also at −24-25 retention time as the major immuno-reactive peak and at −28 min retention time. Thus, the authentic presence of Opiorphin in milk has been assessed after SepPak cartridge extraction of samples in the presence of EDTA, a purification procedure that eliminates the ELISA-interfering milk-components. According to these extraction conditions, the physiological concentration range of Opiorphin in human milk was established at 27±3 ng/ml, n=9 determinations (in duplicate) for one sample (n °2-06).

### Sequences

Nucleic acid encoding the BPLP protein (SEQ ID No 1):

| | |
|---|---|
| aattgagtat ctggcaagag taagattaag cagtaatttg ttccaaagaa gaatcttcta | 60 |
| ccaaggagca actttaaaga atg aaa tta act ttc ttc ttg ggc ctg ttg gct | 113 |
| Met Lys Leu Thr Phe Phe Leu Gly Leu Leu Ala | |
| 1 5 10 | |
| 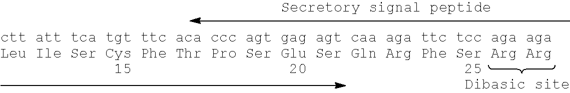 | 161 |
| cca tat cta cct ggc cag ctg cca cca cct cca ctc tac agg cca aga | 209 |
| Pro Tyr Leu Pro Gly Gln Leu Pro Pro Pro Pro Leu Tyr Arg Pro Arg | |
| 30 35 40 | |
| tgg gtt cca cca agt ccc cca cct ccc tat gac tca aga ctt aat tca | 257 |
| Trp Val Pro Pro Ser Pro Pro Pro Pro Tyr Asp Ser Arg Leu Asn Ser | |
| 45 50 55 | |
| cca ctt tct ctt ccc ttt gtc cca ggg cga gtt cca cca tct tct ttc | 305 |
| Pro Leu Ser Leu Pro Phe Val Pro Gly Arg Val Pro Pro Ser Ser Phe | |
| 60 65 70 75 | |
| tct cga ttt agc caa gca gtc att cta tct caa ctc ttt cca ttg gaa | 353 |
| Ser Arg Phe Ser Gln Ala Val Ile Leu Ser Gln Leu Phe Pro Leu Glu | |
| 80 85 90 | |
| tct att aga caa cct cga ctc ttt ccg ggt tat cca aac cta cat ttc | 401 |
| Ser Ile Arg Gln Pro Arg Leu Phe Pro Gly Tyr Pro Asn Leu His Phe | |
| 95 100 105 | |
| cca cta aga cct tac tat gta gga cct att agg ata tta aaa ccc cca | 449 |
| Pro Leu Arg Pro Tyr Tyr Val Gly Pro Ile Arg Ile Leu Lys Pro Pro | |
| 110 115 120 | |
| ttt cct cct att cct ttt ttt ctt gct att tac ctt cct atc tct aac | 497 |
| Phe Pro Pro Ile Pro Phe Phe Leu Ala Ile Tyr Leu Pro Ile Ser Asn | |
| 125 130 135 | |
| cct gag ccc caa ata aac atc acc acc gca gat aca aca atc acc aca | 545 |
| Pro Glu Pro Gln Ile Asn Ile Thr Thr Ala Asp Thr Thr Ile Thr Thr | |
| 140 145 150 155 | |
| aat ccc ccc acc act gca aca gca acc acc agg cac ttc cac aaa acc | 593 |
| Asn Pro Pro Thr Thr Ala Thr Ala Thr Thr Arg His Phe His Lys Thr | |
| 160 165 170 | |
| cac aat gac gat cag ctc ctc aac agt acc tat ctc ttc aac acc aga | 641 |
| His Asn Asp Asp Gln Leu Leu Asn Ser Thr Tyr Leu Phe Asn Thr Arg | |
| 175 180 185 | |
| gcc tgc cac ctc cat atc agc agc aac ccc cgc agc atc tac tga | 686 |
| Ala Cys His Leu His Ile Ser Ser Asn Pro Arg Ser Ile Tyr | |
| 190 195 200 | |
| aaatactact caaattctcg ccaaccgtcc tcacacagta ttgctcaatg ccactgtcca | 746 |
| agttacgact tccaaccaaa ctatattaag cagcccagcc tttaaaagtt tttggcaaaa | 806 |
| actctttgcc atttttggtt gaacatgcaa taaatgatat tttccaaact gctctgatat | 866 |
| cttagaagaa ataaactgca atgattttga tggaaccaac cctgatctaa ccagcacact | 926 |
| aaataaagta tttgagcaat a | 947 |

```
BPLP protein (SEQ ID No 2):
Met Lys Leu Thr Phe Phe Leu Gly Leu Leu Ala Leu Ile Ser Cys Phe
    1 5 10 15

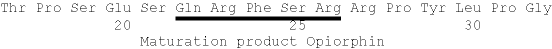

Gln Leu Pro Pro Pro Pro Leu Tyr Arg Pro Arg Trp Val Pro Pro Ser
                  35 40 45
Pro Pro Pro Pro Tyr Asp Ser Arg Leu Asn Ser Pro Leu Ser Leu Pro
          50 55 60
Phe Val Pro Gly Arg Val Pro Pro Ser Ser Phe Ser Arg Phe Ser Gln
  65 70 75 80
Ala Val Ile Leu Ser Gln Leu Phe Pro Leu Glu Ser Ile Arg Gln Pro
                                  85 90 95
Arg Leu Phe Pro Gly Tyr Pro Asn Leu His Phe Pro Leu Arg Pro Tyr
                        100 105 110
Tyr Val Gly Pro Ile Arg Ile Leu Lys Pro Pro Phe Pro Pro Ile Pro
                115 120 125
Phe Phe Leu Ala Ile Tyr Leu Pro Ile Ser Asn Pro Glu Pro Gln Ile
        130 135 140
Asn Ile Thr Thr Ala Asp Thr Thr Ile Thr Thr Asn Pro Pro Thr Thr
145 150 155 160
Ala Thr Ala Thr Thr Arg His Phe His Lys Thr His Asn Asp Asp Gln
                                165 170 175
Leu Leu Asn Ser Thr Tyr Leu Phe Asn Thr Arg Ala Cys His Leu His
                        180 185 190
Ile Ser Ser Asn Pro Arg Ser Ile Tyr
                195 200
```

1-34. (canceled)

35. A method for prognosis, diagnosis, or monitoring of a pathological state in a subject comprising:
a) measuring a quantitative level of a BPLP gene product, in a biological sample obtained from the subject;
b) comparing the quantitative level of the BPLP gene product measured in a) to a reference value of the BPLP gene product;
wherein a significantly higher or lower level of the BPLP gene product compared to the reference value is an indication for the prognosis, diagnosis, or monitoring of the pathological state.

36. The method of claim 35, wherein the diagnosis is carried out and further comprises at least one selected from the group consisting of:
determining that the pathological state is linked with the BPLP gene product in a subject; and
confirming that the pathological state is linked with the BPLP gene product in a subject.

37. The method of claim 35, wherein the significantly higher or lower level of the BPLP gene product compared to the reference value is an indication that the pathological state is linked with the BPLP gene product in a subject.

38. The method of claim 35, further comprising:
designing a treatment regimen for the subject.

39. The method of claim 35, wherein the biological sample is selected from the group consisting of blood, plasma, saliva, urine, cerebrospinal fluid, at least one tear, sperm, and milk.

40. The method of claim 35, wherein the BPLP gene product is BPLP protein, a peptide derived from the BPLP protein, a BPLP precursor protein, or a maturation product of the BPLP protein.

41. The method of claim 40, wherein the maturation product is present and is the QRFSR-peptide Opiorphin of SEQ ID NO: 3.

42. The method of claim 40, wherein the maturation product is present and is
extracted by at least one of acid-methanol extraction and by C18-SepPak cartridge extraction, or
purified by reverse phase C18-HPLC chromatography, or
extracted by at least one of acid-methanol extraction and by C18-SepPak cartridge extraction, and purified by reverse phase C18-HPLC chromatography.

43. The method of claim 35, wherein the quantitative level of the BPLP gene product is measured with an immuno-assay.

44. The method of claim 43, wherein the immuno-assay is a competitive ELISA.

45. The method of claim 43, wherein the immuno-assay comprises a polyclonal anti-QRFSR antibody (‘QRFSR’ disclosed as SEQ ID NO: 3).

46. The method of claim 35, wherein the sample is a tissue and the BPLP gene product is mRNA.

47. A kit for a prognostic, diagnostic, or monitoring test, wherein the kit comprises:
a binding agent for specifically recognizing a BPLP gene product;
at least one reagent to detect the binding of the agent with the BPLP gene product; and
optionally, at least one selected from the group consisting of a positive control sample and a negative control sample.

48. The kit of claim 47, wherein the BPLP gene product is a BPLP-protein maturation product.

49. The kit of claim 48, wherein the BPLP-protein maturation product is the QRFSR-peptide Opiorphin of SEQ ID NO: 3.

50. The kit of claim 47, wherein the binding agent is an antibody or a molecular probe.

51. A method of producing a polyclonal antibody specifically binding to the QRFSR-peptide Opiorphin of SEQ ID NO: 3 comprising:
a) administering a peptide of SEQ ID NO: 4, conjugated in a covalent manner to a ovalbumin-(CO—NH)— molecule, to a non-human animal; and
b) selecting a polyclonal antibody specifically binding to the QRFSR-peptide (SEQ ID NO: 3).

52. A method for obtaining a hybridoma that secretes an antibody specifically binding to the QRFSR-peptide Opiorphin of SEQ ID NO: 3, comprising:
a) administering a peptide of SEQ ID NO: 4, conjugated in a covalent manner to a ovalbumin-(CO—NH)— molecule, to a non-human animal;
b) removing at least one immunoglobulin-secreting lymphocyte from the non-human animal;
c) fusing the at least one lymphocyte with myeloma cells so as to obtain at least one immunoglobulin-secreting hybridoma;
d) selecting a hybridoma that secretes an antibody specifically binding to the QRFSR-peptide (SEQ ID NO: 3); and
e) optionally, isolating the antibody.

53. An antibody specifically binding to a QRFSR-peptide Opiorphin of SEQ ID NO: 3 obtained by the method of claim 51.

54. An antibody specifically binding to a QRFSR-peptide Opiorphin of SEQ ID NO: 3 obtained by the method of claim 52.

55. A method for detecting a QRFSR-peptide Opiorphin of SEQ ID NO: 3 in a sample susceptible to comprising the Opiorphin, the method comprising:
a) coating a micro-titration plate with a peptide of SEQ ID NO 4 in which a 6-polyethylene or a 12-polyethylene linker has been introduced between the first and the second residues;
b) adding the sample and the polyclonal antibody of claim 52;
c) adding a labelled antibody directed against the polyclonal antibody;
d) adding a chromogenic substrate suitable for detecting the labelled antibody; and
e) detecting a signal emitted by the chromogenic substrate,
thereby detecting Opiorphin in the sample susceptible to comprising Opiorphin.


SEQUENCE LISTING (9 sequences)

SEQ ID NO: 1 - DNA, 947 bp, Homo sapiens
  CDS (81)..(683)
  sig_peptide (81)..(143)
  misc_feature (144)..(158): Region encoding a maturation product of opiorphin
aattgagtatctggcaagagtaagattaagcagtaatttgttccaaagaagaatcttctaccaaggagcaactttaaagaatgaaattaactttcttcttgggcctgttggctcttatttcatgtttcacacccagtgagagtcaaagattctccagaagaccatatctacctggccagctgccaccacctccactctacaggccaagatgggttccaccaagtcccccacctccctatgactcaagacttaattcaccactttctcttccctttgtcccagggcgagttccaccatcttctttctctcgatttagccaagcagtcattctatctcaactctttccattggaatctattagacaacctcgactctttccgggttatccaaacctacatttcccactaagaccttactatgtaggacctattaggatattaaaacccccatttcctcctattcctttttttcttgctatttaccttcctatctctaaccctgagccccaaataaacatcaccaccgcagatacaacaatcaccacaaatccccccaccactgcaacagcaaccaccaggcacttccacaaaacccacaatgacgatcagctcctcaacagtacctatctcttcaacaccagagcctgccacctccatatcagcagcaacccccgcagcatctactgaaaatactactcaaattctcgccaaccgtcctcacacagtattgctcaatgccactgtccaagttacgacttccaaccaaactatattaagcagcccagcctttaaaagtttttggcaaaaactctttgccatttttggttgaacatgcaataaatgatattttccaaactgctctgatatcttagaagaaataaactgcaatgattttgatggaaccaaccctgatctaaccagcacactaaataaagtatttgagcaata

SEQ ID NO: 2 - protein, 201 aa, Homo sapiens
MKLTFFLGLLALISCFTPSESQRFSRRPYLPGQLPPPPLYRPRWVPPSPPPPYDSRLNSPLSLPFVPGRVPPSSFSRFSQAVILSQLFPLESIRQPRLFPGYPNLHFPLRPYYVGPIRILKPPFPPIPFFLAIYLPISNPEPQINITTADTTITTNPPTTATATTRHFHKTHNDDQLLNSTYLFNTRACHLHISSNPRSIY

SEQ ID NO: 3 - protein, 5 aa, Homo sapiens
QRFSR

SEQ ID NO: 4 - protein, 6 aa, Artificial Sequence
  Description of Artificial Sequence Synthetic peptide
YQRFSR

SEQ ID NO: 5 - protein, 6 aa, Artificial Sequence
  Description of Artificial Sequence Synthetic peptide
YQRFSR

SEQ ID NO: 6 - protein, 5 aa, Artificial Sequence
  Description of Artificial Sequence Synthetic peptide
QRFSR

SEQ ID NO: 7 - protein, 5 aa, Rattus norvegicus
QHNPR

SEQ ID NO: 8 - protein, 5 aa, Artificial Sequence
  Description of Artificial Sequence Synthetic peptide
QRFSR

SEQ ID NO: 9 - protein, 6 aa, Artificial Sequence
  Description of Artificial Sequence Synthetic peptide
YQRFSF